(19) United States
(12) Patent Application Publication
(10) Pub. No.: US 2025/0041100 A1
(43) Pub. Date: Feb. 6, 2025

(54) EXTERNAL CATHETER ASSEMBLY

(71) Applicant: Tanya Taylor Edwards, Clarksdale, MS (US)

(72) Inventor: Tanya Taylor Edwards, Clarksdale, MS (US)

(21) Appl. No.: 18/229,990

(22) Filed: Aug. 3, 2023

(51) Int. Cl. A61F 5/453; A61F 5/44; A61F 5/443

(52) U.S. Cl. CPC A61F 5/453; A61F 5/4401; A61F 5/443

(57) ABSTRACT

An external catheter assembly for improved incontinence care for immobile or bedridden patients includes a catheter sleeve and a flexible material enclosed within the catheter sleeve. The catheter sleeve may include an insertion end and a tapered end. The catheter sleeve has an internal cavity extending into the catheter sleeve and outwardly through the insertion end. The internal cavity being configured to receive a penile shaft of a user. The flexible material enclosed within the catheter sleeve provides a filling configured to absorb a fluid evacuated through the penile shaft. A drainage tube is attached to the tapered end. The drainage tube is in fluid communication with the flexible material and is configured to drain the fluid out of the catheter sleeve.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to catheters and more particularly pertains to a new catheter for improved incontinence care for immobile or bedridden patients.

### (2) Description of Related Art Including Information Disclosed Under 37 CFR 1.97 and 1.98

The prior art relates to catheters. The prior art discloses diaper-like incontinence products and internal catheter devices. Many of the prior art devices, systems, and assemblies that require frequent changing or that can cause complications such as skin breakdown, bedsores, and urinary tract infections (UTIs). Thus, there is a need in the prior art for a non-invasive catheter device that can be used in medical facilities, inpatient hospice facilities, nursing homes, and patient's homes.

### BRIEF SUMMARY OF THE INVENTION

An embodiment of the disclosure meets the needs presented above by generally comprising a catheter sleeve and a flexible material enclosed within the catheter sleeve. The catheter sleeve may include an insertion end and a tapered end. The catheter sleeve has an internal cavity extending into the catheter sleeve and outwardly through the insertion end. The internal cavity being configured to receive a penile shaft of a user. The flexible material enclosed within the catheter sleeve provides a filling configured to absorb a fluid evacuated through the penile shaft. A drainage tube is attached to the tapered end. The drainage tube is in fluid communication with the flexible material and is configured to drain the fluid out of the catheter sleeve.

There has thus been outlined, rather broadly, the more important features of the disclosure in order that the detailed description thereof that follows may be better understood, and in order that the present contribution to the art may be better appreciated. There are additional features of the disclosure that will be described hereinafter, and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of an external catheter assembly according to an embodiment of the disclosure.

FIG. 2 is a front view of an embodiment of the disclosure.

FIG. 3 is a back view of an embodiment of the disclosure.

FIG. 4 is a side view of an embodiment of the disclosure.

FIG. 5 is a cross-sectional, in-use view of an embodiment of the disclosure.

### DETAILED DESCRIPTION OF THE INVENTION

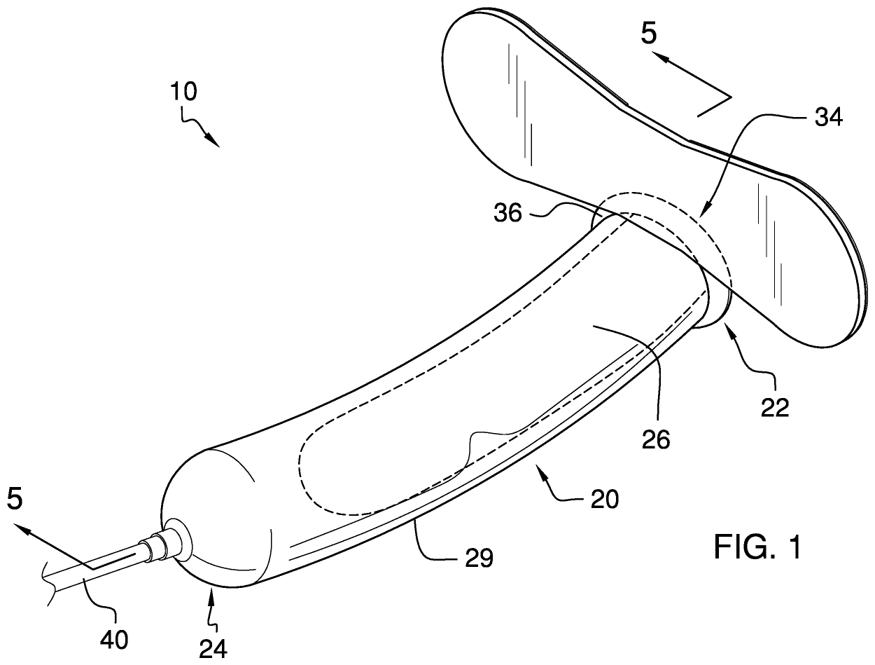

With reference now to the drawings, and in particular to FIGS. 1 through 5 thereof, a new catheter embodying the principles and concepts of an embodiment of the disclosure and generally designated by the reference numeral 10 will be described.

As best illustrated in FIGS. 1 through 5, the external catheter assembly 10 generally comprises a catheter sleeve 20, a flexible material 30 enclosed within the catheter sleeve 20, and a drainage tube 40 that is attached to the catheter sleeve 20 and in fluid communication with the flexible material 30.

The catheter sleeve 20 generally includes an insertion end 22 and a tapered end 24. Generally, the insertion end 22 is opposite the tapered end 24. The catheter sleeve 20 has an internal cavity 26 extending into the catheter sleeve 20 and outwardly through the insertion end 22. The internal cavity 26 is configured to receive a penile shaft 70 of a user. The catheter sleeve 20 may be tubular to further accommodate the penile shaft 70.

Embodiments of the catheter sleeve 20 further comprise a mesh layer 28 that is surrounded by an outer layer 29, which may also be referred to herein as a peripheral wall 29. In embodiments, the peripheral wall 29 may be formed of a durable, latex-free plastic material.

The internal cavity 26 extends within the mesh layer 28, so that the mesh layer 28 surrounds the penile shaft 70. In some embodiments, the mesh layer 28 may be formed of a soft cloth material. The mesh layer 28 may also be flexible and capable of curving to mirror the shape of the penile shaft 70 of a particular user. In other embodiments, the catheter sleeve 20 itself may be pre-formed in shapes resembling a banana, half-moon, or other tubular shape configured to mirror the shape of the penile shaft 70. For example, embodiments may be produced in a range of sizes and shapes to accommodate a range of penile shaft 70 sizes and shapes.

The flexible material 30 generally defines a filling configured to absorb a fluid 74 evacuated through the penile shaft 70. The flexible material 30 is enclosed within the catheter sleeve 20 between the internal cavity 26 and the peripheral wall 29. In certain embodiments, the tapered end 24 may further comprise a collection area 32 that extends between the mesh layer 28 and the outer peripheral wall 29. For example, the flexible material 30 may be more densely packed within the collection area 32 than in the rest of the catheter sleeve 20. The collection area 32 is generally configured to draw the fluid 74 away from the penile shaft 70 and toward the tapered end 24.

Embodiments of the insertion end 22 further include a soft roll opening 34 extending inwardly into the internal cavity 26. The soft roll opening 34 further extends through the peripheral wall 29 and through the mesh layer 28. The soft roll opening 34 is configured to receive the penile shaft 70, such that the penile shaft 70 may be housed within the internal cavity 26. Particular embodiments of the soft roll opening 34 also include a supplementary length 36 of the peripheral wall 29. The supplementary length 36 is rolled up around a circumference of the soft roll opening 34. The supplementary length 36 is configured to expand a diameter of the soft roll opening 34 to receive a testicle 72 of the user. For example, some users may experience improved fit and stability of the external catheter assembly 10 when both the penile shaft 70 and the testicles 72 are inserted within the catheter sleeve 20.

An adhesive member 38 may also be attached to the outer layer 39 proximate the soft roll opening 34. The adhesive member 38 is configured to secure and stabilize the catheter sleeve 20 in place when the soft roll opening 34 has received the penile shaft 70. The adhesive member 38 has a sticky surface configured to safely and comfortably secure the catheter sleeve 20 onto the user. In preferred embodiments, the adhesive member 38 can stick to the skin of the user while the user is using the catheter sleeve 20 and comfortably peel off of the skin of the user once the user is finished with the catheter sleeve 20. Ideally, placement and removal of the adhesive member on the skin will not cause the skin to tear or break down.

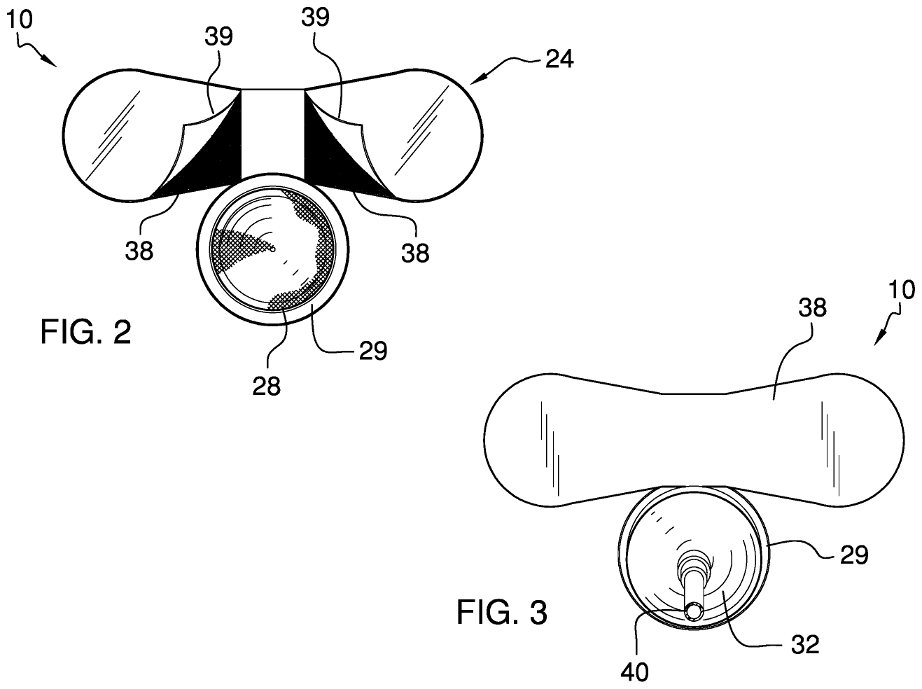

As shown in FIG. 2, the adhesive member 38 may further include a peel-off layer 39 that covers the adhesive member 38 before the user places the catheter sleeve 20 on the body. The peel-off layer 39 is generally configured to protect the adhesive member 38 from getting dirty, worn, or otherwise degraded until the catheter sleeve 20 is in place and the peel-off layer 39 is removed to expose the adhesive member 38. In exemplary embodiments, the peel-off layer is formed of paper, cloth, or other fibrous materials.

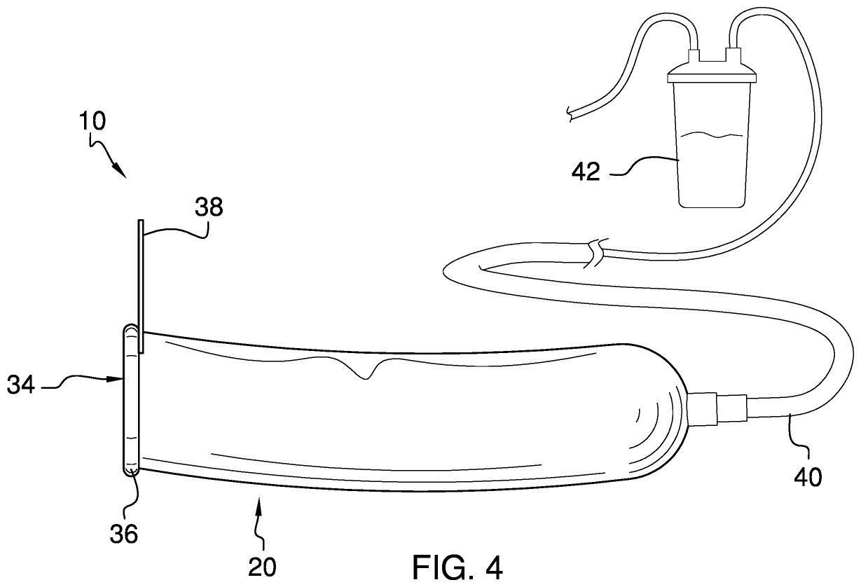

The drainage tube 40 is attached to the tapered end 24. The drainage tube is in fluid communication with the flexible material 30 and is configured to drain the fluid 74 out of the catheter sleeve 20. In preferred embodiments, the drainage tube 40 is in fluid communication with the flexible material 30 that is located within the collection area 32. Some embodiments of the drainage tube 40 may also be configured for attachment to and fluid communication with a collection canister 42, as shown in FIG. 4. For example, the collection canister 42 may provide a suction force to pull the fluid 74 out of the collection area 32 through the drainage tube 40. The user can then collect the fluid 74 within the collection canister 42, for example for sanitary disposal.

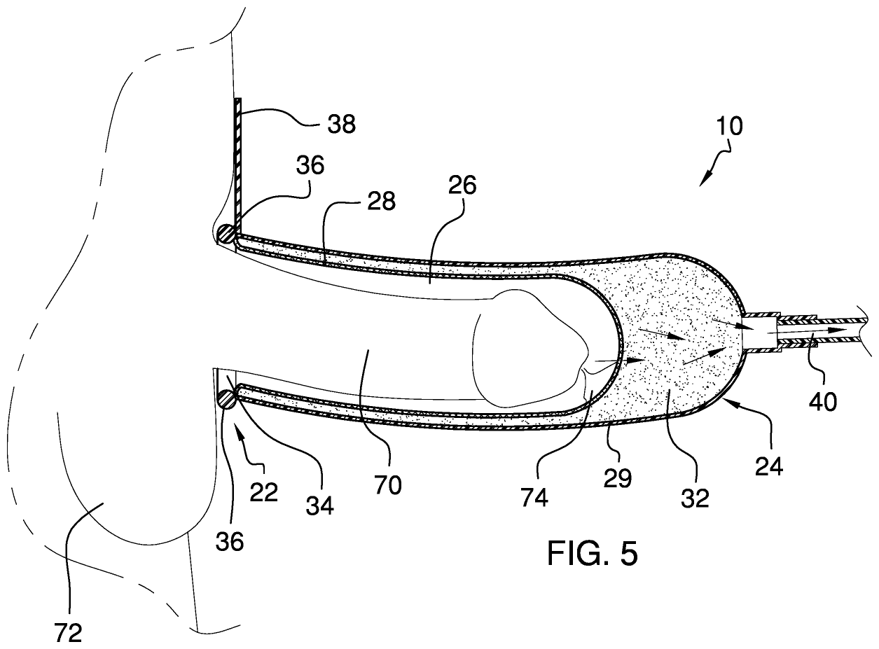

In use, as shown by FIG. 5, a user may insert the penile shaft 70 into the internal cavity 26 through the insertion end 22. When fluid 74 is expelled by the penile shaft 70, it may be absorbed by the flexible material 30 surrounding the mesh layer 28. At least partially because there is a greater density of absorbent, flexible material 30 at the collection area 32, and because the collection area 32 is situated proximate where fluid 74 is likely to be expelled by the penile shaft 70, the fluid 74 will pool into the collection area 32. As shown by the small arrows in FIG. 5, the fluid 74 can then drain out of the catheter sleeve 20 through the drainage tube 40. If the drainage tube 40 is connected to a collection canister 42 capable of providing suction, the fluid 74 may be more efficiently drained out of the catheter sleeve 20. To secure the position of the catheter sleeve 20, the user can stick the adhesive member 38 to the skin. As shown in FIG. 5, the user may stick the adhesive member 38 to the skin above the penile shaft 70, for example because the user wants to also insert the testicles 72 into the catheter sleeve 20. If so, the supplementary length 36 of the peripheral outer layer 29 can unroll from the soft roll opening 34 to accommodate the testicles 72. In alternative embodiments, the user may instead wish to stick the adhesive member 38 to the skin on either side of the penile shaft 70 or below the penile shaft 70. To change the catheter sleeve 20, the user can remove the adhesive member 38 from the skin and remove the penile shaft 70 from the internal cavity 26.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the elements is present, unless the context clearly requires that there be only one of the elements.

I claim:

1. An external incontinence catheter device, comprising:
a catheter sleeve, the catheter sleeve including an insertion end and a tapered end, the catheter sleeve having an internal cavity extending into the catheter sleeve and outwardly through the insertion end, the internal cavity being configured to receive a penile shaft of a user;
a flexible material enclosed within the catheter sleeve and defining a filling configured to absorb a fluid evacuated through the penile shaft, the flexible material being enclosed within the catheter sleeve between the internal cavity and a peripheral wall of the catheter sleeve; and
a drainage tube being attached to the tapered end, the drainage tube being in fluid communication with the flexible material and being configured to drain the fluid out of the catheter sleeve.

2. The external incontinence catheter device of claim 1, wherein the catheter sleeve is tubular.

3. The external incontinence catheter device of claim 1, the catheter sleeve further comprising:
a mesh layer surrounded by the peripheral wall, the internal cavity extending within the mesh layer.

4. The external incontinence catheter device of claim 3, the mesh layer being formed of a soft cloth material.

5. The external incontinence catheter device of claim 3, the tapered end further comprising:
a collection area extending between the mesh layer and the peripheral wall, the collection area being configured to draw a fluid away from the penile shaft and toward the tapered end.

6. The external incontinence catheter device of claim 5, wherein the collection area is filled by the flexible material.

7. The external incontinence catheter device of claim 5, wherein the drainage tube is in fluid communication with the collection area.

8. The external incontinence catheter device of claim 1, the drainage tube being configured to attach to and be in fluid communication with a collection canister.

9. The external incontinence catheter device of claim 1, wherein the peripheral wall is formed of a durable, latex-free plastic material.

10. The external incontinence catheter device of claim 1, the insertion end further comprising:
a soft roll opening extending inwardly into the internal cavity, the soft roll opening extending through the peripheral wall and through the mesh layer, the soft roll opening being configured to receive the penile shaft, thereby housing the penile shaft within the internal cavity.

11. The external incontinence catheter device of claim 10, the soft roll opening further comprising:
a supplementary length of the peripheral wall, the supplementary length being rolled up around a circumference of the soft roll opening, the supplementary length being configured to expand a diameter of the soft roll opening to receive a testicle of the user.

12. The external incontinence catheter device of claim 10, further comprising:
an adhesive member being attached to the peripheral wall proximate the soft roll opening, the adhesive member being configured to secure the catheter sleeve in place when the soft roll opening has received the penile shaft.

13. The external incontinence catheter device of claim 12, the adhesive member further comprising:
a peel-off layer removably covering the adhesive member, the peel-off layer being configured to protect the adhesive member from degradation until the catheter sleeve is in place and the peel-off layer is removed to expose the adhesive member.

14. The external incontinence catheter device of claim 13, wherein the peel-off layer is formed of paper, cloth, or other fibrous materials.

15. An external incontinence catheter system, comprising:
a collection canister;
a removable external catheter device, the removable external catheter device comprising:
a catheter sleeve, the catheter sleeve including an insertion end and a tapered end, the catheter sleeve having an internal cavity extending into the catheter sleeve and outwardly through the insertion end, the internal cavity being configured to receive a penile shaft of a user;
a flexible material enclosed within the catheter sleeve and defining a filling configured to absorb a fluid evacuated through the penile shaft, the flexible material being enclosed within the catheter sleeve between the internal cavity and a peripheral wall of the catheter sleeve; and
a drainage tube being attached to the tapered end, the drainage tube being in fluid communication with the flexible material and being configured to drain the fluid out of the catheter sleeve.

16. An external male incontinence catheter device, comprising:
a catheter sleeve, the catheter sleeve including an outer layer surrounding a mesh layer, the catheter sleeve having a tapered end opposite an insertion end, the mesh layer having an internal cavity extending through the insertion end, the outer layer being formed of a durable, latex-free plastic material, the mesh layer being formed of a soft cloth material, the catheter sleeve being tubular;
the insertion end having a soft roll opening extending inwardly into the internal cavity, the soft roll opening extending through the outer layer and through the mesh layer, the soft roll opening being configured to receive a penile shaft of a user, thereby housing the penile shaft within the internal cavity, the soft roll opening having a supplementary length of the outer layer, the supplementary length being rolled up around a circumference of the soft roll opening, the supplementary length being configured to expand the soft roll opening to receive a testicle of the user;
an absorbent material enclosed within the catheter sleeve between the outer layer and the mesh layer;
the tapered end having a collection area extending between the mesh layer and the outer layer, the collection area being filled by the absorbent material, the collection area being configured to draw a fluid away from the penile shaft and toward the tapered end;
a drainage tube being attached to the outer layer at the tapered end, the drainage tube being in fluid communication with the collection area, the drainage tube being configured to attach to and be in fluid communication with a collection canister, the drainage tube being configured to drain the fluid out of the collection area and to allow for suction of the fluid through the drainage tube into the collection canister; and
an adhesive member being attached to the outer layer proximate the soft roll opening, the adhesive member being configured to secure the catheter sleeve in place when the soft roll opening has received the penile shaft.
